(19) United States
(12) Plant Patent Application Publication
(10) Pub. No.: US 2015/0020253 P1
(43) Pub. Date: Jan. 15, 2015

(54) Mandevilla plant named 'HOTLIPS'

(50) ;

(71) Applicant: Keep Smiling B.V., 's-Gravenzande (NL)

(72) Inventor: Eelco van Staalduinen, 's-Gravenzande (NL)

(21) Appl. No.: 13/987,204

(22) Filed: Jul. 11, 2013

(51) Int. Cl. A01H 5/00

(52) U.S. Cl. PLT/232

(57) ABSTRACT

A new and distinct cultivar of Mandevilla sanderi plant named ‘HOTLIPS’, characterized by a vigorous plant with glossy leaves, many buds and flowers, an intense dark red color of the flowers and continuous flowering.

### LATIN NAME OF GENUS AND SPECIES OF THE PLANT CLAIMED

Mandevilla sanderi

### VARIETY DENOMINATION

‘HOTLIPS’

### BACKGROUND OF THE INVENTION

The present invention relates to a new and distinct cultivar of Mandevilla plant, botanically known as Mandevilla sanderi (Hemsl.) Woodson, commonly known as Brazilian Jasmine, and hereinafter referred to by the name ‘HOTLIPS’.

The new Mandevilla ‘HOTLIPS’ is a product of a planned breeding program conducted by the inventor, Eelco van Staalduinen, in Almeria, Spain. The objective of the breeding program is to develop a new Mandevilla cultivar with 1. a vigorous plant; 2. glossy leaves; 3. many buds and flowers; 4. intense dark red color of the flowers; and 5. continuous flowering.

The new Mandevilla cultivar originated from a naturally-occurring, whole plant mutation of the Mandevilla variety, ‘DIPLADENIA SANDERI RED’ (unpatented).

The new Mandevilla variety ‘HOTLIPS’ was discovered and selected by the inventor as a single flowering plant within a planting of the stated mutation in a controlled environment in 2008 in Almeria, Spain.

Asexual reproduction of the new Mandevilla cultivar by cuttings was first performed in 2009 in Almeria, Spain, and has demonstrated that the combination of characteristics as herein disclosed for the new cultivar are firmly fixed and retained through successive generations of asexual reproduction. The new cultivar reproduces true-to-type.

### SUMMARY OF THE INVENTION

The following traits have been repeatedly observed and are determined to be the unique characteristics of ‘HOTLIPS’. These characteristics in combination distinguish ‘HOTLIPS’ as a new and distinct cultivar:

1. Vigorous plant

2. Glossy leaves

3. Many buds and flowers

4. Intense dark red color of the flowers

5. Continuous flowering.

Of the many commercial cultivars known to the present inventor, the most similar to the new Mandevilla sanderi ‘Hotlips’ is the parent variety ‘DIPLADENIA SANDERI RED’, and differs from the new cultivar Mandevilla sanderi ‘Hotlips’ in the following characteristics described in Table 1:

TABLE 1
Comparison with Parent/Similar Variety

| | | Comparison Cultivar |
|---|---|---|
| | | ‘DIPLADENIA |
| | New Cultivar | SANDERI RED’ |
| Characteristic | ‘HOTLIPS’ | (unpatented) |
| Flower colour | Dark intense red | Red |
| Flower colour | Dark intense red | Pale red |
| at high | | |
| temperatures | | |

### BRIEF DESCRIPTION OF THE PHOTOGRAPHS

The accompanying colored photographs illustrate the overall appearance of the new Mandevilla sanderi cultivar ‘HOTLIPS’, showing the colors as true as it is reasonably possible to obtain in colored reproductions of this type. Colors in the photographs may differ slightly from the color values cited in the detailed botanical description, which more accurately describe the actual colors of ‘HOTLIPS’.

FIG. 1 shows a typical plant of ‘Hotlips’.

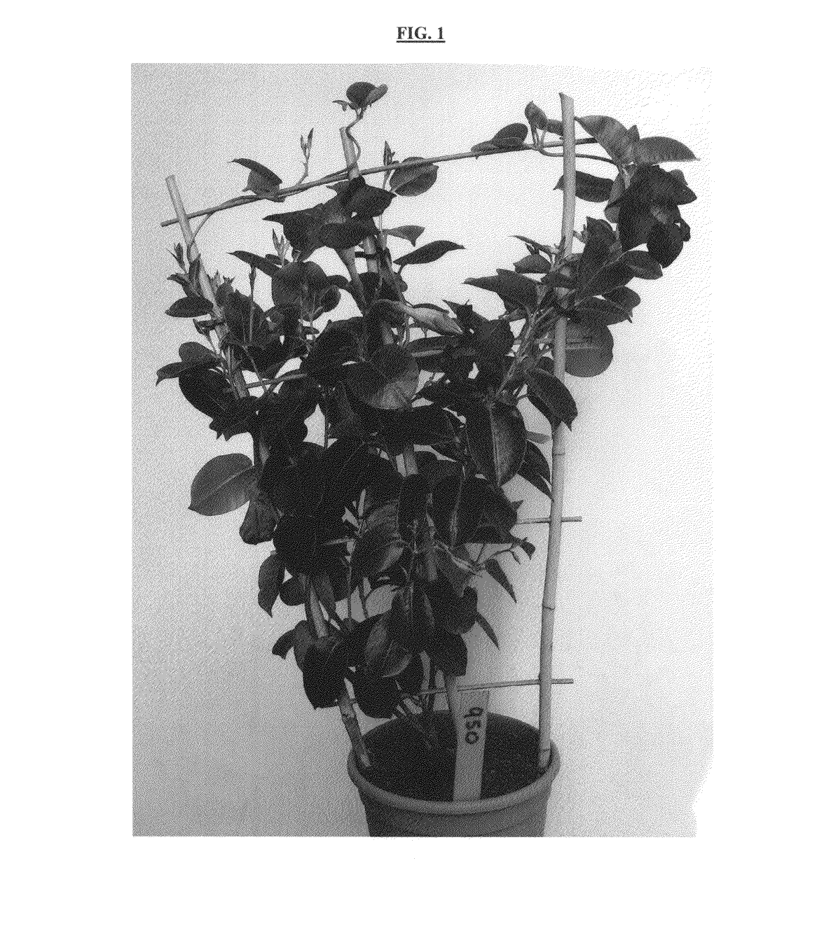

### DETAILED BOTANICAL DESCRIPTION

The new Mandevilla ‘HOTLIPS’ has not been observed under all possible environmental conditions. The phenotype of the new cultivar may vary significantly with variations in environment such as temperature, light intensity, day length, and fertility level without any variance in genotype.

The aforementioned photographs, together with the following observations, measurements and values describe plants of the new Mandevilla ‘HOTLIPS’ as grown under conditions which closely approximate those generally used in commercial practice.

The plant grows preferably in Mediterranean climate conditions in greenhouses or plastic covers.

The age of the ‘HOTLIPS’ plants described is 6 months.

Color references are made to The Royal Horticultural Society Color Chart (R.H.S.), 4^{th} edition, except where general colors of ordinary significance are used.
Classification:
   Botanical.—Mandevilla sanderi.
   Variety denomination.—HOTLIPS.
Parentage: Naturally occurring mutation of ‘DIPLADENIA Sanderi Red’
Propagation:
   Type.—By cuttings.
Plant description:
   Form.—Twiner.
   Vigor.—Strong.
   Plant height.—Medium: 40 cm.
   Plant spread (diameter).—Medium: 35 cm.
   Lateral branches.—Color: Medium Green: RHS 134A Anthocyanin Coloration: Absent.
Foliage description:
   Arrangement.—Opposite.
   Length.—About 71 mm.
   Width.—About 49 mm.
   Overall shape.—Elliptic; recurving in longitudinal cross-section.
   Apex shape.—Acuminate.
   Base shape.—Slightly cordate.
   Texture.—Upper Surface: Medium to Strong Glossiness Lower Surface: Medium Glossiness.
   Pubescence.—None.
   Color.—Mature: Upper surface: Dark Green RHS 135A; Lower Surface: Medium Green RHS 141c.
   Petiole.—Length: About 16 mm Diameter: About 2 mm Texture: Strong glossiness Color: RHS 143C Anthocyanin Coloration: Absent Pubescence: Weak.
Inflorescence description:
   Type.—Racemose.
   Natural flowering season.—May-October.
   Flower longevity.—Medium.
   Fragrance.—Weak.
   Number of flowers per inflorescence.—1.
   Flower shape.—Acute.
   Flower diameter.—5-6 cm.
   Flower depth (height).—5 mm.
   Corolla shape.—Funnelform.
   Corolla diameter.—About 77 mm.
   Tube length.—About 24 mm.
   Tube texture.—Smooth.
   Tube color.—Light green with a weak purple-red flush RHS N144C.
   Throat length.—About 32 mm.
   Throat width.—About 19 mm.
   Throat texture.—Smooth.
   Throat color.—Basal, outer side: Yellow-Green, RHS 150D Basal, inner side: Orange, Between RHS 28A and RHS 28B Distal, outer side: Light Yellow, RHS 11D, fading into main color (dark red RHS 53A) Distal, inner side: Orange, between RHS 28A and RHS 28B, fading into light yellow-orange RHS 23C Limb shape: Asymmetric; convex in longitudinal cross-section Limb tip: Acuminate Limb margin: weak to medium undulation Limb color: Upper side: Dark red, RHS 53A Lower side: Dark red, RHS 53A Peduncles: Length: 6 cm Diameter: 4 mm Texture: Glossy Strength: Strong Color: Light green RHS 143B Pedicels: Length: 1.5 cm Diameter: 3 mm Color: Light Green RHS 143B Anthocyanin Coloration: None. Pubescence: None.
   Buds.—Shape: Obtrullate. Length: 2.5 cm Diameter: 6 mm Color: RHS N144A.
   Sepals.—Arrangement: symmetric Quantity per flower: 5 Length: 3 cm Width: 3 cm Color Basal half: Medium Green RHS 143B Distal half: Light Green with Red pointed lobes 143D.
Reproductive organs:
   Androecium.—Stamen: Quantity: 5 Anther: Color: Light Yellow RHS 4D Filament Color: Light Green RHS 143D.
   Gynoecium.—Ovary: Color: Medium Green RHS 143B
Seed: none observed
Weather tolerance: not tested
Disease/pest resistance: not tested
Disease/pest susceptibility: not tested

I claim:

1. A new and distinct variety of Mandevilla plant named ‘HOTLIPS’, as illustrated and described herein.
